(19) United States
(12) Patent Application Publication
(10) Pub. No.: US 2024/0294968 A1
(43) Pub. Date: Sep. 5, 2024

(54) SYSTEMS AND METHODS FOR VERIFYING BIOLOGICAL SAMPLES WITH WHOLE GENOME SEQUENCING

(71) Applicant: HudsonAlpha Institute for Biotechnology, Huntsville, AL (US)

(72) Inventors: Josh P. Clevenger, Huntsville, AL (US); Walid Korani, Madison, AL (US)

(73) Assignee: HudsonAlpha Institute for Biotechnology, Huntsville, AL (US)

(21) Appl. No.: 18/594,521

(22) Filed: Mar. 4, 2024

Related U.S. Application Data
(60) Provisional application No. 63/449,934, filed on Mar. 3, 2023.

(51) Int. Cl. C12Q 1/6809; G16B 20/20

(52) U.S. Cl. CPC C12Q 1/6809; G16B 20/20; C12Q 2600/13

(57) ABSTRACT

The present disclosure is directed to methods for verifying the genetic makeup of a target product, for example, products in a supply chain. The methods relate to the use of genomics for confirming or verifying the genetic makeup of the target product. The methods also relate to the use of genomics for detecting the presence or absence of one or more gene variants associated with a specific phenotypic quality or characteristic in the target product. The presently disclosed method verifies and/or detects certain genetic markers of the target product using whole genome sequencing (WGS) to access the entire genome.

### CROSS REFERENCE TO RELATED APPLICATIONS

This application claims the benefit of and priority to U.S. Provisional Application No. 63/449,934, filed on Mar. 3, 2023, and entitled “Systems and Methods for Verifying Ingredients,” the disclosure of which is expressly incorporated by reference in its entirety.

### FIELD OF THE DISCLOSURE

The present disclosure relates generally to methods and kits for verifying the genetic makeup of a sample, and more particularly, for identifying a set of biomarkers using whole genome sequencing to verify the genotype of biological samples.

### BACKGROUND

Genome sequencing, or reading DNA, has matured for use in everyday applications and industries, including understanding family relationships, learning the breeds of pet cats and dogs, and identifying criminals. Geneticists read DNA to learn how diversity across genomes affects the development of organisms and their interaction with the environment. Methods for reading DNA vary based on the parts of DNA read, such as specific changes in DNA or a sampling of the genome. Whole genome sequencing (WGS) is a technique that reads all of an organism's DNA. WGS generates enough sequence to read each letter, or base pair, of a target genome numerous times. For example, human genomes for clinical use are sequenced to an average depth of 30 reads per base pair. This “genome coverage,” or the amount of sequence generated needed to cover a target genome×number of times, directly impacts the time and expense of reading the DNA of a single individual.

Depending on the size of the genome and the coverage needed for analysis, WGS can be cost-prohibitive. Current traditional high throughput genotyping technologies generally utilize fixed marker arrays or only sample small portions of the full genome to be cost efficient. In addition to the sequencing cost, data analysis to find differences between individuals or between specific breeds or cultivars is challenging as varieties in the same species often share genetic material. For example, in the context of plants, a particular challenge is when in a population of plants there exists more than one species of a genus of the plant, where one or more species has a characteristic distinct from the other yet is morphologically indistinct. An example of such a situation is where within a population of plants, the wild type species is inter-planted with another species that is more aggressive, more resistant to herbicide application, or has another undesirable characteristic. Detecting low abundant mutations in the genetic material to differentiate these species can be difficult and often becomes an obstacle when attempting to identify species or varieties within the same species, especially if the sequencing is run on low depth.

Accordingly, there remains a need in the art for a low-cost, highly efficient method to verify one or more biomarkers of a particular species within a genus and/or a particular variety within the species using short sequencing on low depth.

### SUMMARY

The problems expounded above, as well as others, are addressed by the following inventions, although it is to be understood that not every embodiment of the inventions described herein will address each of the problems described above.

In some embodiments, a method of verifying the genetic makeup of an organic material is provided, the method including obtaining a biological sample of the organic material; obtaining a reference sequence from a verified organic material; performing whole genome sequencing on the biological sample to generate one or more target sequence reads; and comparing the sequences of the one or more target sequence reads to the reference sequence, wherein the genetic makeup of the organic material is verified when the one or more target sequence reads is substantially similar to that of the reference sequence.

In one embodiment, the reference sequence includes a profile of one or more single nucleotide polymorphism variants that are present within the verified organic material. In another embodiment, the one or more target sequence reads is substantially similar to that of the reference sequence when the one or more target sequence reads includes at least 90 percent identity to the reference sequence. In still another embodiment, the one or more target sequence reads is substantially similar to that of the reference sequence when the one or more target sequence reads includes the one or more of the single polymorphism variants. In another embodiment, the whole genome sequencing is performed at a depth of about 1× or less. In yet another embodiment, the method may further include labeling the organic material as genetically similar to the verified organic material when the genetic makeup of the organic material is verified. In another embodiment, the profile of the reference sequence includes one single polymorphism variant every 1 to 5K base pairs (bp) over the length of the genome of the verified organic material.

In further embodiments, a method of verifying the genetic makeup of an organic material is provided, the method including obtaining a biological sample of the organic material; obtaining a pool of verified organic materials; performing whole genome sequencing on each verified organic material from the pool; identifying one or more single nucleotide polymorphism variants that are present within the verified organic materials and extracting any falsely identified single nucleotide polymorphisms to generate a reference sequence; performing whole genome sequencing on the biological sample to generate one or more target sequence reads; comparing the sequences of the one or more target sequence reads to the reference sequence; and verifying the genetic makeup of the organic material when the one or more target sequence reads is substantially similar to that of the reference sequence.

In one embodiment, the pool of verified organic materials is comprised of heterozygous species. In this embodiment, the pool includes four or more samples of verified organic materials. In another embodiment, the pool of verified organic materials is comprised of homozygous species. In this embodiment, the pool includes ten or more samples of verified organic materials. In still another embodiment, the reference sequence is comprised of a profile including one single polymorphism variant every 1 to 5K base pairs (bp) over the length of the genome of the verified organic material. In yet another embodiment, the method includes labeling the organic material as genetically similar to the verified organic material when the genetic makeup of the organic material is verified. In still another embodiment, the one or more target sequence reads is substantially similar to that of the reference sequence when the one or more target sequence reads comprises at least 90 percent identity to the reference sequence. For example, the one or more target sequence reads is substantially similar to that of the reference sequence when the one or more target sequence reads comprises at least 95 percent identity to the reference sequence. In another embodiment, the organic material is a plant or an agricultural product.

### BRIEF DESCRIPTION OF THE DRAWINGS

Further features and advantages can be ascertained from the following detailed description that is provided in connection with the drawings described below:

FIG. 1 shows a graphical representation of exemplary data obtained by the methods of the present disclosure according to one embodiment.

FIG. 2 is a graphical representation showing the results of a validation experiment for verifying the genetic makeup of single peanut variety according to one embodiment of the present disclosure.

FIG. 3 is a graphical representation showing the results of a validation experiment for verifying the genetic makeup of single peanut variety according to another embodiment of the present disclosure.

FIG. 4 is a graphical representation showing the results of a validation experiment for verifying the genetic makeup of a “CarmEct” variety of cannabis according to one embodiment of the present disclosure.

FIG. 5 is a graphical representation showing the results of a validation experiment for verifying the genetic makeup of an “Anka” variety of cannabis according to one embodiment of the present disclosure.

FIG. 6A is graphical representation showing in silico testing set combinations for verifying the genetic makeup of a pool of peanut varieties according to one embodiment of the present disclosure.

FIGS. 6B and 6C are graphical representations showing the eval values for the testing set combinations of FIG. 6A.

FIG. 7 is a schematic diagram of a computing device for use with the present methods according to one embodiment of the present disclosure.

### DETAILED DESCRIPTION

Unless otherwise defined, all terms (including technical and scientific terms) used herein have the same meaning as commonly understood by one of ordinary skill in the art of this disclosure. It will be further understood that terms, such as those defined in commonly used dictionaries, should be interpreted as having a meaning that is consistent with their meaning in the context of the specification and should not be interpreted in an idealized or overly formal sense unless expressly so defined herein. Well known functions or constructions may not be described in detail for brevity or clarity. All patents, patent applications, published applications and publications, GenBank sequences, databases, websites and other published materials referred to throughout the entire disclosure herein, unless noted otherwise, are incorporated by reference in their entirety.

Following long-standing patent law convention, the terms “a” “an”, and “the” refer to “one or more” when used in this application, including the claims. Thus, for example, reference to “a cell” includes a plurality of such cells, and so forth.

The term “substantially” allows for deviations from the descriptor that do not negatively impact the intended purpose. Descriptive terms are understood to be modified by the term “substantially” even if the word “substantially” is not explicitly recited.

The terms “comprising,” “including,” “having,” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc.

The terms “about” and “approximately” shall generally mean an acceptable degree of error or variation for the quantity measured given the nature or precision of the measurements. For biological systems, the term “about” refers to an acceptable standard deviation of error, preferably not more than 2-fold of a given value. Numerical quantities given herein are approximate unless stated otherwise, meaning that the term “about” or “approximately” can be inferred when not expressly stated.

For the purposes of the present disclosure, the term “gene” has its meaning as understood in the art. However, it will be appreciated by those of ordinary skill in the art that the term “gene” has a variety of meanings in the art, some of which include gene regulatory sequences (e.g., promoters, enhancers, etc.) and/or intron sequences, and others of which are limited to coding sequences. As used herein, the term “gene” can generally refer to a portion of a nucleic acid that encodes a protein. The term can optionally encompass regulatory sequences. The word “gene” can also include references to nucleic acids that do not encode proteins but rather encode functional RNA molecules such as transfer RNAs (tRNAs), ribosomal RNAs (rRNAs), small RNAs (including, but not limited to microRNAs, siRNAs, piRNAs, snoRNAs, snRNAs, exRNAs, scaRNAs) and the long noncoding RNAs. For the purpose of clarity, as used in the present application, the term “gene” generally refers to a portion of a nucleic acid that encodes a protein. This definition is not intended to exclude application of the term “gene” to non-protein coding expression units but rather to clarify that, in most cases, the term as used herein refers to a protein coding nucleic acid.

A gene product or expression product is, in general, an RNA transcribed from the gene or a polypeptide encoded by an RNA transcribed from the gene. Expression of a gene can be measured by a variety of techniques known in the art. Certain techniques can make use of a polynucleotides corresponding to part or all the gene rather than an antibody that binds to a polypeptide encoded by the gene. Appropriate techniques include, but are not limited to, in situ hybridization, Northern blot, and various nucleic acid amplification techniques such as PCR, quantitative PCR, and the ligase chain reaction.

The present disclosure provides methods and kits for verifying the genetic makeup of a target product. The methods of the present disclosure are particularly useful for verifying the genetic makeup of target products that are a species within a genus and/or a particular variety within a species. In some embodiments, the methods relate to the use of genomics for confirming or verifying the genetic makeup of a target product in a supply chain. For example, the methods of the present disclosure can be used to map genes or quantitative trait loci (QTL) in inbred, non-inbred, non-controlled, self-pollinated, out-crossed, or natural populations. In further embodiments, the methods relate to the use of genomics for detecting the presence or absence of one or more gene variants associated with a specific phenotypic quality or characteristic in the target product. In one embodiment, the presently disclosed method verifies and/or detects certain genetic markers of the target product using whole genome sequencing (WGS), for example, low coverage WGS, to access the entire genome. Deploying WGS in accordance with the present disclosure can enhance decision-making, reduce cost and risk, and increase stability and profitability within all supply chains that rely on biological products.

In some embodiments, the methods of the present disclosure can be used to verify the genetic makeup of a target product that is suspected of or otherwise at risk of being mis-identified or subject to genetic drift. The target product may be any product having organic material. In one embodiment, the target product may be a plant or a part thereof. A “plant” may refer to plant tissues, including, for instance, the root, leaf, stem, flower, fruit, seed, and mixtures thereof. In one embodiment, the plant is an agricultural plant. In another embodiment, the target product may be an agricultural product, such as a source of timber or lumber. In another embodiment, the target product may be a food product or feed material. For example, the food product may be any food product that is intended for human or animal consumption. In another embodiment, the food product may be fresh produce, fresh food, frozen food, processed food, a meat product, a dairy product, and mixtures thereof. In still another embodiment, the target product may include a supplement or other ingestible material, such as a nutritional supplement. In yet another embodiment, the target product may be a medical or pharmaceutical product, a building material, a textile product, a cosmetic product, a biofuel, an essential oil, a fertilizer, or any combination thereof.

In one embodiment, the method includes obtaining a biological sample from the target product that is to be verified. As used herein, a “biological sample” refers to any sample containing DNA that is derived from the target product. Examples of a biological sample include, but are not limited to, a tissue, plant matter, a cell sample, cellular structures, genetic material, or a combination thereof. The biological sample can be provided or obtained in any form known in the art. By way of non-limiting example, the biological sample can be formalin-fixed paraffin-embedded (FFPE) tissue, fresh frozen (FF) samples, freeze-dried samples, raw samples, processed samples, dehydrated samples, unsprouted seeds, sprouted seedlings, samples from “off-the-shelf” food products, samples from one or more ingredients to be included in a food product, or other sample types.

The biological sample can be obtained by a variety of conventional techniques. As used herein, the phrase, “obtaining a biological sample,” refers to any process for directly or indirectly acquiring or generating the biological sample from the target product. For example, the biological sample can be obtained from any point in the supply chain. The biological sample can be obtained from a field, a factory, a farm, a seed supplier, a laboratory facility, a manufacturer, a distributor, a retailer, or any other point in the supply chain.

In one embodiment, the methods of the present disclosure include measuring or determining the expression level of one or more genes from the biological sample and comparing the expression level of the one or more genes in the biological sample to that of a verified target product. As used herein, the term “verified target product” refers to a product whose identity has been verified or is known. A “verified target product” can be obtained directly from an individual within a species or group of individuals within the species that are genetically isolated from exposure to another individual or a separate group of individuals of the same species. A “verified target product” can include organic material from one or more individuals of a particular species that are known to have the given phenotypic quality or characteristic that is to be determined in the test sample. For example, the “verified target product” may be a specific variety of plant, a reference based on outbound seeds to individual farms or growing areas, a historical variety of plant, a specific or historical variety of a grain, a specific or historical variety of a nut, and so forth.

In this embodiment, the methods of the present disclosure include a step of generating a reference sequence (referred to herein as the “reference panel”). The methods utilize de novo sequencing, which refers to sequencing a novel genome where there is no reference sequence available. The reference panel can be generated by sequencing random biological samples from a pool of verified target products. In highly diverse or heterozygous species, the reference panel can be generated by sequencing as low as four samples. For instance, in highly diverse or heterozygous species, the reference panel can be generated by sequencing at least four samples. In another embodiment, in highly diverse or heterozygous species, the reference panel can be generated by sequencing at least five samples. In still another embodiment, in highly diverse or heterozygous species, the reference panel can be generated by sequencing at least six samples. In still another embodiment, in highly diverse or heterozygous species, the reference panel can be generated by sequencing at least seven samples. On the other hand, in low diverse or high homozygous species, a higher number of samples may be needed for generating the reference panel. For example, in low diverse or high homozygous species, at least ten samples may be needed to generate the reference panel. In one embodiment, in low diverse or high homozygous species, the reference panel can be generated by sequencing at least eleven samples. In still another, in low diverse or high homozygous species, the reference panel can be generated by sequencing at least twelve samples. In yet another embodiment, in low diverse or high homozygous species, the reference panel can be generated by sequencing at least fifteen samples.

In one embodiment, the methods of the present disclosure involve sequencing, for instance, skim-sequencing, a number of the biological samples in the pool of verified target products (referred to as the “control samples”). The sequencing is performed at a certain sequencing depth. In the context of the present disclosure, sequencing depth, or coverage, refers to the average number of sequencing reads that align to, or cover, known reference bases. The sequencing coverage level (i.e., depth) typically determines whether variant discovery can be made with a certain degree of confidence at particular base positions. At higher depths, each base is covered by a greater number of aligned sequence reads, which means that base calls can be made with a higher degree of confidence. In one embodiment of the present disclosure, the sequencing for generating the reference panel is low coverage sequencing. Low coverage whole genome sequencing, as described herein, refers to sequencing to about 5× coverage or less, and preferably about 1× coverage or less. For example, the sequencing for generating the reference panel can be performed at a depth of about 1× per sample in the pool.

Based on the sequences obtained from the control samples, the sequences can be compared for common variation. In this embodiment, the reference panel can be created by calling the single nucleotide polymorphisms (SNPs) and calculating the allele frequencies. As used herein, a “single nucleotide polymorphism” (SNP) refers to a genomic variant at a single base position in a DNA sequence and “SNP calling” refers to identifying the variable sites in the sequences.

In one embodiment, the SNP calling step of the present disclosure uses a series of algorithms that efficiently extract falsely identified SNPs from the full panel. The methods include performing a detection algorithm of the biological sample, for instance, detecting one or more SNPs. Certain embodiments employ a binary model algorithm for biological sample classification. Embodiments of the present disclosure provide a method of substantially reducing the number of genes in the reference panel, for example, the falsely identified SNPs, without significantly compromising detection accuracy. In some embodiments, the total number of genes is reduced to less than 50. In certain embodiments, the total number of genes is less than 25. In further embodiments, the total number of genes can be reduced to between 15-20, inclusive. In some embodiments, the total number of genes are reduced to about 1, 2, 3, 4, 5, 6, 7, 8, 9, 10, 11, 12, 13, 14, 15, 16, 17, 18, 19, 20, 21, 22, 23, 24, 25, 26, 27, 28, 29, and about 30. The methods of the present disclosure control the error rate such the sequence error is as low as one nucleotide for every 50,000 nucleotides. In one embodiment, the reference panel may be generated using the computational tool, Khufu, which was developed by the Applicant, HudsonAlpha Institute for Biotechnology

The methods of the present disclosure result in a reference panel that includes a profile of single nucleotide polymorphism (SNP) variants for the verified target product. In one embodiment, to represent the entire genome, the reference panel includes one SNP every 1 to 5K base pairs (bp) over the length of the genome. In another embodiment, the reference panel may include one SNP every 1K bp over the length of the genome. In still another embodiment, the reference panel may include one SNP every 2K bp over the length of the genome. In yet another embodiment, the reference panel may include one SNP every 3K bp over the length of the genome. In another embodiment, the reference panel may include one SNP every 4K bp over the length of the genome. In yet another embodiment, the reference panel may include one SNP every 5K bp over the length of the genome.

Once the reference panel is established, the genetic makeup of the target product can be assessed by conducting whole genome sequencing of the biological sample to generate one or more target sequence reads and comparing the target sequence reads to the reference panel. In one embodiment, whole genome sequencing can be performed on an individual biological sample from a target product (referred to as the “test sample”) and the resulting sequence can be compared to the reference panel. In another embodiment, the method may include performing whole genome sequencing on a plurality of biological samples from a sample pool of target products (referred to as the “test sample pool”) and comparing the generated sequences to the reference panel, as will be described in more detail below.

In some embodiments, the whole genome sequencing performed on the test sample or test sample pool is low coverage, as defined above. For example, the test sample or test sample pool can be sequenced at a depth of about 1× coverage or less. In addition, while whole genome sequencing has been described herein for generating one or more target sequence reads of the control and test samples, other methodology for analyzing a large number of nucleic acids in a single reaction can be utilized. For instance, the methods may employ other high-throughput nucleic acid sequencing technologies, such as next generation sequencing (NGS). In one embodiment, a targeted RNA-sequence panel can be offered on an NGS platform. Non-limiting examples of such NGS technologies include instruments and protocols from Illumina, Inc (San Diego, CA, USA), Thermo Fischer Scientific (Waltham, MA, USA), Qiagen (Venlo, Netherlands).

In still other embodiments, certain multiplex PCR-based platforms are employed. Embodiments may use qPCR-based platforms configured to accommodate a substantial number of analytes, such as the Qiagen Modaplex (Venlo, Netherlands) or similar platform. Embodiments can also employ conventional real time PCR platforms. The term “PCR” or “polymerase chain reaction” as used herein refers to the use of template DNA, nucleotides (dNTPS) and primers that bind to the template DNA to selectively amplify a target sequence. PCR is a technology that can be used to amplify a single copy or few copies of a DNA sequence by several orders of magnitude, generating thousands to millions of copies of the DNA sequence. Standard PCR methods are known in the art. PCR amplification and the detection of an amplified target sequence can be used to detect specific gene elements in a sample. Quantitative PCR methods such as real time PCR may be used to determine the absolute or relative amounts of a known sequence in a sample. Digital PCR methods may also be used for detecting and/or quantifying target sequences in a sample.

In further embodiments, any of various expression microarray-based platforms can be utilized, including, but not limited to those from Affymetrix (Santa Clara, CA, USA), Aknonni Biosystems (Frederick, MD, USA), Biofire Diagnostics (Salt Lake City, UT, USA), or similar platforms. Some embodiments may employ non-array platforms and protocols. In certain non-array embodiments, a quantitative nuclease protection assay is utilized. Other non-array embodiments employ platforms and protocols from NanoString Technologies, Inc. (Seattle, WA, USA). The examples provided herein are merely exemplary and should not be considered limiting in any way. Embodiments employ any of various molecular diagnostic platforms.

Certain embodiments may employ a DNA chip, which is a device that is convenient to compare expression levels of a number of genes at the same time. DNA chip-based expression profiling can be carried out, for example, by the method as disclosed in “Microarray Biochip Technology” (Mark Schena, Eaton Publishing, 2000). A DNA chip comprises immobilized high-density probes to detect a number of genes. Thus, expression levels of many genes can be estimated at the same time by a single round analysis. Namely, the expression profile of a specimen can be determined with a DNA chip.

In some embodiments, a difference in the allele frequency between the one or more target sequence reads from the test sample or test sample pool and the reference panel can be assessed to determine whether the genetic makeup of the target product is substantially similar to that of the verified target product. In this embodiment, the test sample or test sample pool can be run using a SNP calling module. In one embodiment, the test sample or test sample pool is run using the SNP calling module, KhufuVAR, which is the Khufu variant caller. Similar to the SNP calling step for generating the reference panel, the SNP calling step for generating the target sequence uses the series of algorithms that efficiently extract falsely identified SNPs and accurately identify the SNPs correlated to a given trait in the target product. The relative allele frequency, as compared to the reference panel, can then be averaged across all loci (after dropping any missing data) to obtain a measured allele frequency. The measured allele frequencies of each sample can be compared with the reference panel.

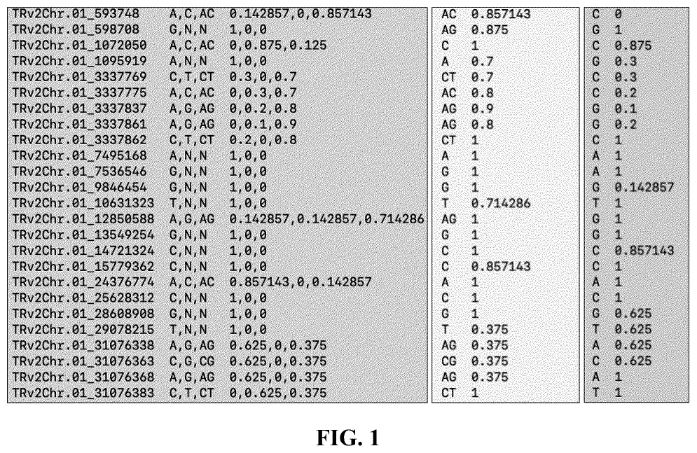

FIG. 1 is a graphical representation of exemplary data obtained by the methods of the present disclosure for use in assessing the similarity between the target sequence reads from the one or more test samples and the reference panel. As shown in FIG. 1, the graphical representation includes three panels: a left panel, a middle panel, and a right panel. The left panel shows data obtained from the reference panel. The first column within the left panel shows each loci in the reference panel, the second column shows the recognized allele structures, and the third column shows the calculated allele frequencies. The middle panel and the right panel show two different test samples with each of the first columns showing the recognized allele structures and the second columns showing the calculated allele frequencies.

In one embodiment, the genetic makeup of the target product is determined to be substantially similar to the genetic makeup of the verified target product if the allele frequency similarity between the target sequence read and the reference panel is within an acceptable degree of variance. Statistical methodology may be employed to determine an acceptable degree of variance. For example, in one embodiment, a subset of control samples can be run among the test samples as a positive control and the T-test can be used to differentiate between the target product and non-target products. The “T-test” is an inferential statistic used to determine if there is a significant difference between the means of two groups and how they are related. In some embodiments, the T-test may not be necessary when a reasonable number of test samples are available and/or the estimated diversity among the overall sample pool is large. If, on the other hand, a low number of test samples are available or the target product is being compared with a close relative, the T-test may be needed. Statistical significance can also be determined by calculating the number of standard deviations from the mean that constitute a positive or negative result.

In some embodiments, the one or more target sequence reads can be determined to be substantially similar to that of the reference panel when the one or more target sequence reads includes at least 90 percent identity to the reference sequence panel. In another embodiment, the one or more target sequence reads can be determined to be substantially similar to that of the reference panel when the one or more target sequence reads includes at least 95 percent identity to the reference sequence panel. In still another embodiment, the one or more target sequence reads can be determined to be substantially similar to that of the reference panel when the one or more target sequence reads includes at least 98 percent identity to the reference sequence panel. In yet another embodiment, the one or more target sequence reads can be determined to be substantially similar to that of the reference panel when the one or more target sequence reads includes at least 99 percent identity to the reference sequence panel. In still another embodiment, the one or more target sequence reads can be determined to be substantially similar to that of the reference panel when all of the calls for the SNPs in the panel are identical.

By comparing the sequences of the target product to the reference panel in accordance with the methods of the present disclosure, the genetic makeup of the target product can be confirmed against the verified target product. For instance, in one embodiment, the methods can predict the likelihood that the target product is or is not the same species as the verified target product. In another embodiment, the methods can predict the likelihood that the target product is or is not the same variety of species as the verified target product. In further embodiments, the methods of the present disclosure can also distinguish one variety of a species from another variety of the same species.

In some embodiments, the results and information obtained from the methods of the present disclosure can also be used to label a target product with information pertaining to its genetic makeup. For example, the method may confirm that the target product is the same species or variety of species as the verified target product. In this aspect, the method may include labelling the target product as genetically similar or identical to the verified target product. For instance, the target product may be genetically certified and labelled the same. In contrast, the method may confirm that the target product is not the same species or variety of species as the verified target product. In this aspect, the method may include labelling the target product to indicate that it is not genetically similar to the verified target product.

In further embodiments, the methods of the present disclosure can be used to identify one or more biomarkers, such as an allele, associated with a specific phenotypic quality or characteristic that are uniquely present in the target product and verified target product. In certain embodiments, gene expression profiles are determined for a specific panel of genes that are present within the verified target product using the methods described herein. The biological sample can then be examined for the presence or absence of one or more biomarkers by performing the methods disclosed herein. For instance, in the context of agricultural products, the biomarkers may be associated with qualities or characteristics such as the ability of the product to resist and tolerate disease, pests, and/or stress. Hence, it can be useful to determine whether the biomarker of interest is present or absent in the target product and at what expression level.

In still further embodiments, by determining the presence or absence of one or more biomarkers in the target product, the methods of the present disclosure can be used to make improvements to the supply chain. For example, the methods of the present disclosure can treat or prevent disease in a geographic region where agricultural products are grown and that is susceptible to and/or experiencing infestation of the disease. By identifying a target product that has a biomarker present for resistance/tolerance to the disease, the target product can be cultivated in the geographic region. As another example, the methods can increase crop yield in the geographic region affected by disease by planting or cultivating the target product having the disease-resistant biomarker. In still further embodiments, the methods can prevent genetic drift in a geographic region. For instance, by determining the presence or absence of one or more biomarkers in the target product, genetic drift can be quickly identified and isolated, which allows a grower/supplied to replant the area with drift or manage the area more aggressively to prevent disease.

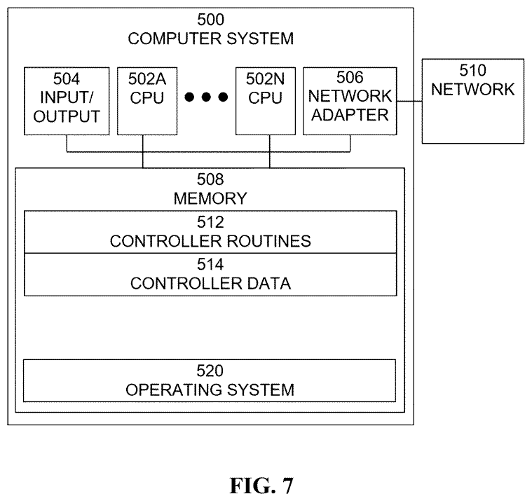

FIG. 7 is a schematic device of a computing device 500 for use with the methods of the present disclosure. The computing device 500 may be implemented using one or more programmed general-purpose computer systems, such as embedded processors, systems on a chip, personal computers, workstations, server systems, and minicomputers or mainframe computers, or in distributed, networked computing environments. The computing device 500 may include one or more processors (CPUs) 502A-502N, input/output circuitry 504, network adapter 506, and memory 508. CPUs 502A-502N execute program instructions to carry out the functions of the present systems and methods. Typically, CPUs 502A-502N are one or more microprocessors, such as an INTEL CORE® processor.

Input/output circuitry 504 provides the capability to input data to, or output data from, the computing device 500. For example, input/output circuitry 504 may include input devices, such as a graphical user interface, keyboards, mice, touchpads, trackballs, scanners, and analog to digital converters; output devices, such as display screens, video adapters, monitors, and printers; and input/output devices, such as modems.

Network adapter 506 interfaces the computing device 500 with a network 510. Network 510 may be any public or proprietary data network, such as LAN and/or WAN (for example, the Internet). Memory 508 stores program instructions that are executed by, and data that are used and processed by, CPU 502 to perform the functions of the computing device 500. Memory 508 may include, for example, electronic memory devices, such as random-access memory (RAM), read-only memory (ROM), programmable read-only memory (PROM), electrically erasable programmable read-only memory (EEPROM), and flash memory, and electro-mechanical memory, which may use an integrated drive electronics (IDE) interface, or a variation or enhancement thereof, such as enhanced IDE (EIDE) or ultra-direct memory access (UDMA), or a small computer system interface (SCSI) based interface, or a variation or enhancement thereof, such as fast-SCSI, wide-SCSI, fast and wide-SCSI, or Serial Advanced Technology Attachment (SATA), or a variation or enhancement thereof, or a fiber channel-arbitrated loop (FC-AL) interface.

Memory 508 may include controller routines 512, controller data 514, and operating system 520. Controller routines 512 may include software routines to perform processing to implement one or more controllers configured to perform the methods of the present disclosure. Controller data 514 may include data needed by controller routines 512 to perform processing. In one embodiment, controller routines 512 may include software for generating one or more gene sequences, for instance, performing whole genome sequencing. In another embodiment, controller routines 512 may include software for detecting an SNP. In another embodiment, controller routines 512 may include software for aligning one or more gene sequences. In still another embodiment, controller routines 512 may include software for comparing sequences within the reference panel with one or more target sequence reads from a test sample and determining, based on the comparison, the similarity between the reference panel and the one or more target reads in accordance with the present disclosure.

Moreover, the present disclosure includes kits and reagents for detection, identification, and/or creation of one or more biomarkers or a gene expression profile associated with the verified target product. In some embodiments, the kit includes a means of detecting expression profiles of specific panels of genes. The kit may also include one or more probes.

In some embodiments, the kit can include one or more reagents useful for detection of the level of a gene or gene product in the biological sample. The one or more reagents can be immobilized to a solid support. Non-limiting examples of the composition of the solid support structure include plastic, cardboard, glass, plexiglass, tin, paper, or a combination thereof. The solid support can also include a dip stick, spoon, scoopula, filter paper or swab. In one embodiment, the kit can include a container that contains the one or more reagents.

The reagents can include a labeled compound or agent capable of detecting a gene or gene product (e.g., an scFv or monoclonal antibody) in a biological sample; means for determining the amount of gene expression in the sample; and means for comparing the amount of gene expression in the sample with a standard, such as the verified target product. The compound or agent can be packaged in a suitable container. The kit can further include informational material for using the kit to assess and confirm the genetic makeup of the target product. The informational material can be descriptive, instructional, marketing, or other material that relates to the methods described herein.

In some embodiments, the kit can also include primers for amplifying an mRNA transcribed from a gene that encodes the polypeptide and/or control samples for testing the primers. For example, the control samples can include nucleic acids that hybridize to the primers.

The informational material of the kits is not limited in its form. In one embodiment, the informational material can include information about production of the components of the kit, such as molecular weight, concentration, date of expiration, batch, or production site information, and so forth. In one embodiment, the informational material relates to methods of using the components of the kit. The information can be provided in a variety of formats, include printed text, computer readable material, video recording, or audio recording, or information that provides a link or address to substantive material.

The kit can also include other ingredients, such as solvents or buffers, a stabilizer, or a preservative. Optionally, the kit can include therapeutic agents that can be provided in any form, e.g., liquid, dried or lyophilized form, preferably substantially pure and/or sterile. When the agents are provided in a liquid solution, the liquid solution preferably is an aqueous solution. When the agents are provided as a dried form, reconstitution generally is by the addition of a suitable solvent. The solvent, e.g., sterile water or buffer, can optionally be provided in the kit.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

### Methods and Materials

A data set of a single peanut variety, which was planted on different farms, was used. A random subset of ten samples was used to create the reference panel. The rest of the samples were used as positive controls. Data sets of different accessions from distant peanut varieties of different geographical locations were used as negative controls.

The random subset of ten samples were skim-sequenced at a depth of about 1× per sample in the pool. A reference panel was created by calling SNPs and calculating allele frequencies. The reference panel contained monomorphic, co-dominant, and dominant allele structures and frequencies. The reference panel was created using the computational tool, Khufu. In creating the reference panel with Khufu, the sequence error was as low as one nucleotide for every 50,000 nucleotides, which makes the possibility of calling a wrong allele one time 0.0005. Testing samples were run using a standard SNP calling module. The relative allele frequency, as compared to the reference panel, was averaged across all loci after dropping missing data to obtain an eval value. The eval value of each sample was compared with the others.

### Results

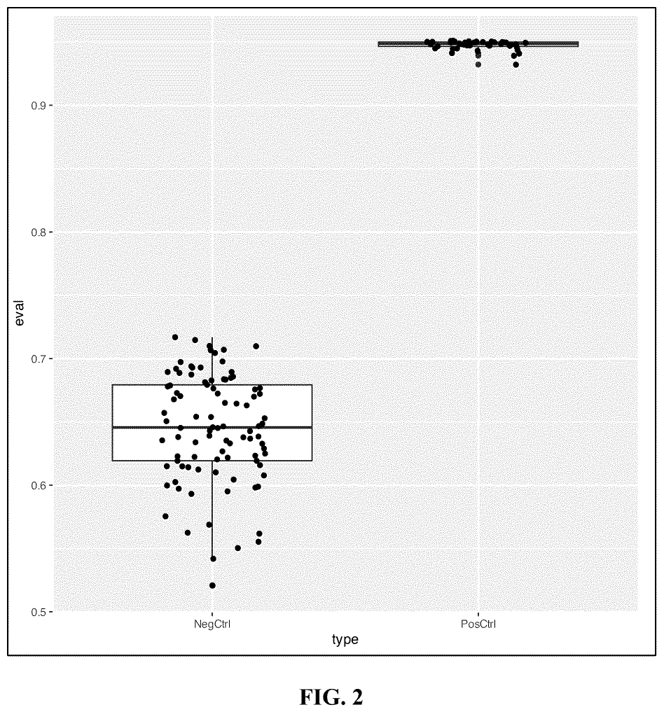

FIG. 2 shows a graphical representation of the results of a validation experiment according to one embodiment. The single peanut variety data set is indicated as the reference panel/positive control and the data set of a first distant peanut accession from a first geographic region is indicated as the negative control. As shown in FIG. 2, since the number of samples in the reference panel was reasonable, a comparison could be made to the distant varieties. The comparison showed that two groups were clear: a group containing the target product and a group containing the distant samples.

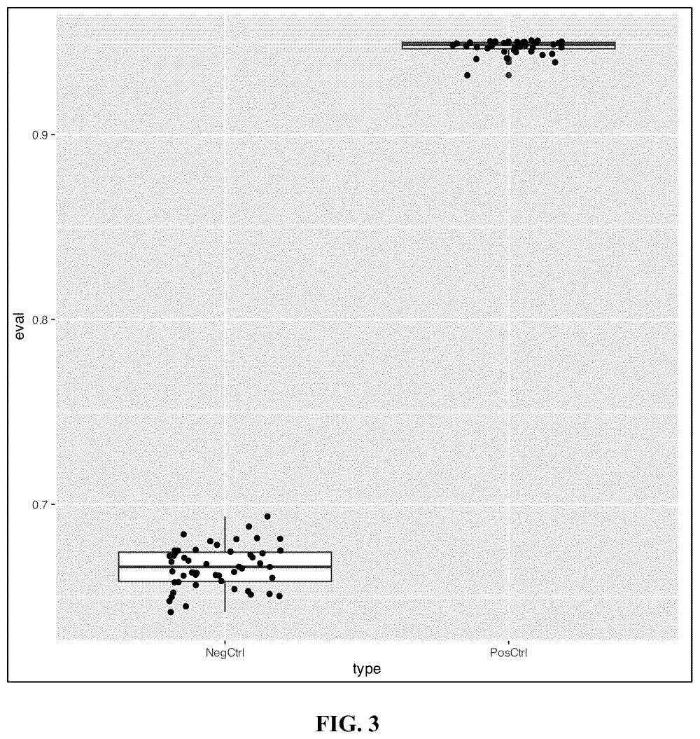

FIG. 3 shows a graphical representation of the results of a validation experiment according to another embodiment. The single peanut variety data set is indicated as the reference panel/positive control and the data set of a second distant peanut accession from a second geographic region is indicated as the negative control. As shown in FIG. 3, the same results were resolved—the testing set was clearly clustered from the positive control.

### Example 2

### Methods and Materials

A data set of cannabis containing eight samples of “CarmEct” variety and the rest were of an unknown variety, was used. Four random samples of the “CarmEct” variety were used to create the reference panel and the other four were used to run positive control and calculate the variance for the T-test. The rest of the samples were used for testing. The same testing method described in Example 1 was used here.

### Results

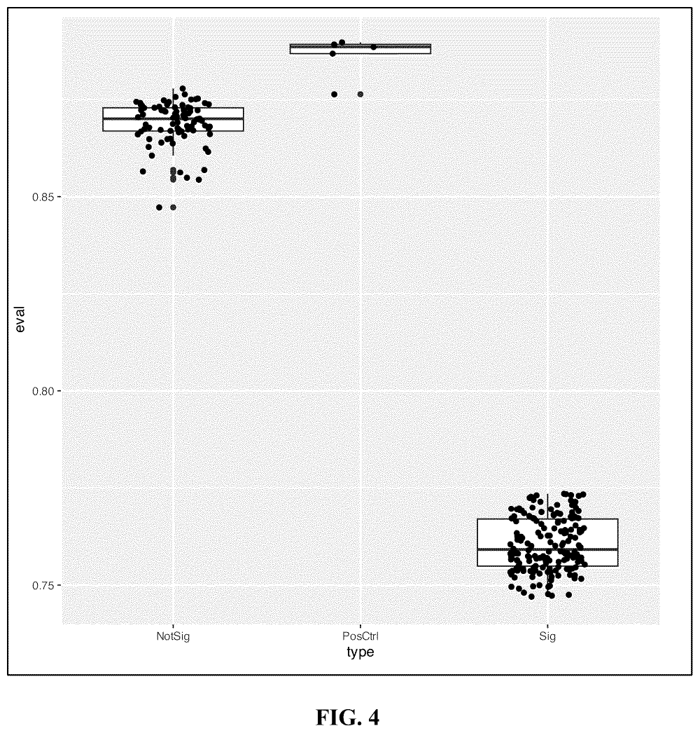

FIG. 4 is a graphical representation showing the results of a validation experiment according to one embodiment. FIG. 4 shows the implementation of the method on the cannabis data set using the “CarmEct” variety where “NotSig” refers to not significant and “Sig” refers to significant based on the T-test. Since the number of samples in the reference panel is lower than in Example 1, the differences between the eval values were smaller so the T-test was needed to find the significant difference among the samples. The testing set was even clustered in two groups based on the P-value of T-test, which distinguishes the true target samples from the different non-target samples.

### Example 3

### Methods and Materials

A data set of cannabis containing eight samples of “Anka” variety and the rest were of an unknown variety, was used. Four random samples of the “Anka” variety were used to create the reference panel and the other four were used to run positive control and calculate the variance for the T-test. The rest of the samples were used for testing. The same testing method described in Example 1 was used here.

### Results

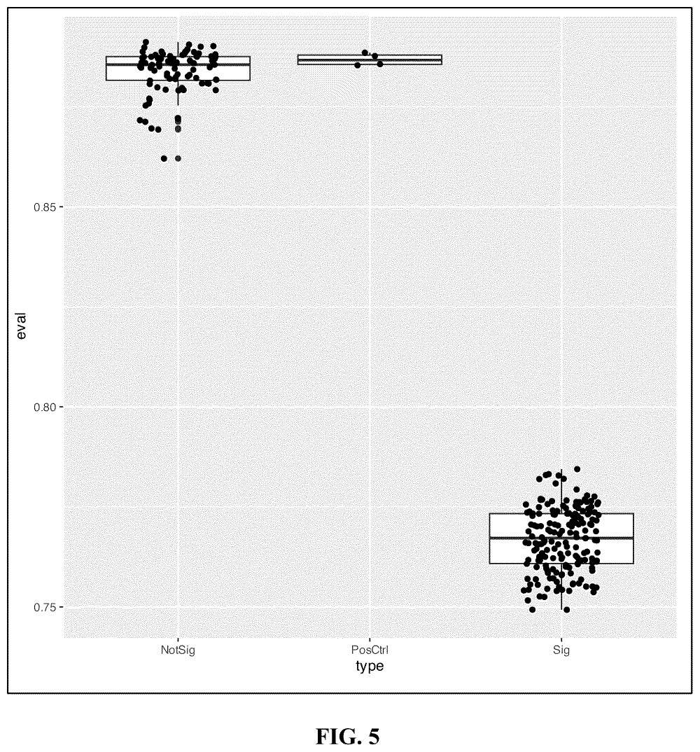

FIG. 5 is a graphical representation showing the results of a validation experiment according to one embodiment. FIG. 5 shows the implementation of the method on the cannabis data set using the “Anka” variety where “NotSig” refers to not significant and “Sig” refers to significant based on the T-test. In this example, the testing set was clustered in two significantly different groups, which distinguishes the true target samples from the different non-target samples.

### Example 4

### Methods and Materials

Samples of the positive control of the reference panel of Example 1 were combined with the two different negative controls described in Example 1 to form serial combinations of in silico pools to validate the methods of the present disclosure. Testing was performed in accordance with the methods of Example 1 to verify the genetic makeup of the pool of peanut varieties.

### Results

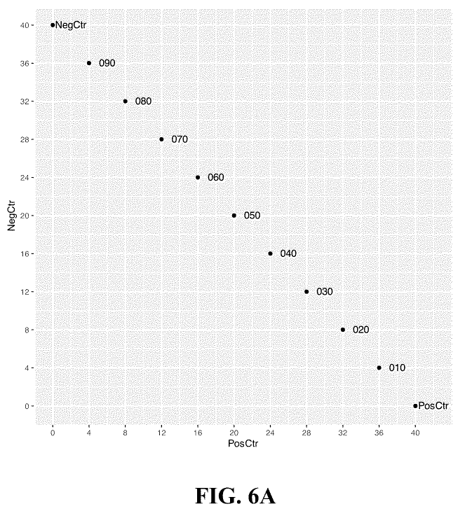

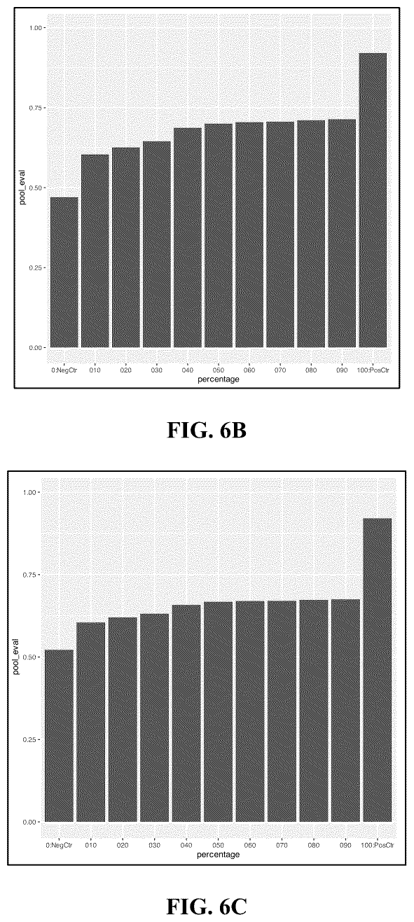

FIG. 6A is a graphical representation showing the in silico testing set combinations of the pool of peanut varieties. FIGS. 6B and 6C are graphical representations showing the eval values for the two different peanut varieties. The first peanut variety from the first geographical region is shown in FIG. 6B and the second peanut variety from the second geographical region is shown in FIG. 6C.

### Prophetic Example 5

Genetic testing can be applied to optimize pine seedlings for growth conditions and thus improve overall yield. For example, seed development organizations produce seeds from specific cultivars for agricultural use. Each cultivar has unique characteristics that affect quality, such as the ability to resist and tolerate disease, pests, or stress. In some cases, growers pay a premium to grow a cultivar with a valuable attribute. They can only capture that value if the purity of the seed meets a specific threshold.

Quality traits in pine are controlled significantly by genetic factors. This has allowed targeted breeding of pine for traits valued by different industries. Seedling suppliers tout plants with genes that provide better quality, faster growth, superior resistance to disease, and higher revenue at harvest. A private landowner planting new tracts invests in genetics to increase the potential for profit at harvest. Decisions on management for the next 25 years will rely on having the best information available. For example, a grower would benefit from knowing if genetic drift has taken place in the planting population. Genetic drift is a change in the frequency of a gene variant in a population due to random chance. It can occur even in the most professional seedling production because plants have evolved to reproduce effectively. Without an understanding of the DNA in a planting population, the way seedlings or young trees look will not illuminate genetic drift that leads to the loss of desired traits in the trees as they grow.

Another concern for growers and manufacturers is “volunteers,” or seeds from the seed bank that grow next to an open canopy. For example, after cutting a tract, seeds in the seed bank can germinate, begin to grow, and potentially outcompete newly planted seedlings. By sampling the newly planted tract for genetic drift, the data will reveal how much, if any, of the seedlings are volunteers.

The methods of the present disclosure can be deployed in the timber industry to use genetics to help reduce risk when planting and harvesting pine forests. The methods can be used to assess newly planted tracts, juvenile tracts, and mature tracts to understand the genetic uniformity of plantings over time. For example, mature tracts can be sampled to provide genetic data to overlay quality data after harvest. In addition, tracts can be sampled and tested for quality traits like microfibril strength, density, and straightness to identify how genetics impact those traits.

The pinus species have extremely large genomes, almost 7 times the size of the human genome. The large genome size has been a limiting factor in deploying genomics effectively for pine. Because the methods of the present disclosure analyze low-coverage WGS, the methods are uniquely suited for species with large genomes. Both loblolly pine (Pinus taeda) and slash pine (Pinus ellottii) have genome sequences available, but a vastly higher-quality pine reference genome can be developed with the methods described herein.

In the present Example, a base population of pine genetics will be established by sampling seedlings purchased from seedling providers. A population of varieties can be sampled to establish a distribution of variation within each variety. These data will provide at least two pieces of information: the extent to which variation occurs within varieties at seedling providers and the extent of diversity between varieties. These data will inform the threshold of distinctness that will inform significant genetic drift within tracts.

DNA can be extracted from needle tissue with high-throughput, high-yield, and high-quality efficiency, and samples can be prepared for sequencing in batches of 96. Sequencing libraries can be prepped by using a Twist Bioscience high-throughput protocol that can be modified to introduce targeted sites. The first set of 96 individuals can be sequenced to 1× coverage (22 Gb, 48 samples per sequencing lane) to introduce a baseline of variation. At least two adjustments can be made after the first 96 samples. First, a panel of variants can be designed that can be used to select for a set of targets in the genome. Second, samples can be sequenced to 0.1× coverage to decrease cost. These adjustments will allow the methods to be used in pine effectively, both in cost and data extracted.

Using this data, it can be established how genetics of trees on a specific plot of land measure up to expectations. The plot of land to be evaluated will have the following criteria: recently planted new seedlings, has areas that are less than 10 years old, and has mature tracts that range from 25 to 50 years old. Then, sampling can occur using two different approaches: (1) sample individuals (96 per defined region in quadrants) and pooled trees (48 pools), and (2) sample each area separately by age. The sampling will explain the genetics of the trees on the plot of land.

Moreover, the data can be used to answer the question of whether overlaying genetic information with quality data taken at a lumber mill allows the modeling of expected quality for unharvested tracts. Simply, if the genetics of a tract of pine is known, can it predict an expected level of quality? Here, tracts that are purchased by industry partners for processing can be sampled. The quality of the lumber can then be compared to the genetics of the forest/plot of land that is logged. The value to the supply chain includes the ability to understand how the genetics correlate to expected quality of the tract. The forestry industry has a significant collection of environmental information that allows them to make decisions. The ability to then add the genetics of the trees will add value to their ability to predict the expected value of the product.

### REFERENCES

Korani, W. et al. (2021). De novo QTL-seq Identifies Loci Linked to Blanchability in Peanut (Arachis hypogaea) and Refines Previously Identified QTL with Low Coverage Sequence. Agronomy 2021, 11(11).
Lou D I, et al. High-throughput DNA sequencing errors are reduced by orders of magnitude using circle sequencing. Proc Natl Acad Sci USA. 2013 Dec. 3; 110(49):19872-7. doi: 10.1073/pnas.1319590110. Epub 2013 Nov. 15. PMID: 24243955; PMCID: PMC3856802.
Van Dijk E. L. et al. Ten years of next-generation sequencing technology. Trends Genet. 30, 418-426. 10.1016/j.tig.2014.07.001.
Cheng C, et al. Methods to improve the accuracy of next-generation sequencing. Front Bioeng Biotechnol. 2023 Jan. 20; 11:982111. doi: 10.3389/fbioe.2023.982111. PMID: 36741756; PMCID: PMC9895957.

The foregoing description illustrates and describes the processes, machines, manufactures, compositions of matter, and other teachings of the present disclosure. Additionally, the disclosure shows and describes only certain embodiments of the processes, machines, manufactures, compositions of matter, and other teachings disclosed, but, as mentioned above, it is to be understood that the teachings of the present disclosure are capable of use in various other combinations, modifications, and environments and is capable of changes or modifications within the scope of the teachings as expressed herein, commensurate with the skill and/or knowledge of a person having ordinary skill in the relevant art. The embodiments described hereinabove are further intended to explain certain best modes known of practicing the processes, machines, manufactures, compositions of matter, and other teachings of the present disclosure and to enable others skilled in the art to utilize the teachings of the present disclosure in such, or other, embodiments and with the various modifications required by the particular applications or uses. Accordingly, the processes, machines, manufactures, compositions of matter, and other teachings of the present disclosure are not intended to limit the exact embodiments and examples disclosed herein. Any section headings herein are provided only for consistency with the suggestions of 37 C.F.R. § 1.77 or otherwise to provide organizational queues. These headings shall not limit or characterize the invention(s) set forth herein.

What is claimed is:

1. A method of verifying the genetic makeup of an organic material, comprising:
obtaining a biological sample of the organic material;
obtaining a reference sequence from a verified organic material;
performing whole genome sequencing on the biological sample to generate one or more target sequence reads; and
comparing the sequences of the one or more target sequence reads to the reference sequence, wherein the genetic makeup of the organic material is verified when the one or more target sequence reads is substantially similar to that of the reference sequence.

2. The method of claim 1, wherein the reference sequence comprises a profile of one or more single nucleotide polymorphism variants that are present within the verified organic material.

3. The method of claim 1, wherein the one or more target sequence reads is substantially similar to that of the reference sequence when the one or more target sequence reads comprises at least 90 percent identity to the reference sequence.

4. The method of claim 2, wherein the one or more target sequence reads is substantially similar to that of the reference sequence when the one or more target sequence reads comprises the one or more of the single polymorphism variants.

5. The method of claim 1, wherein the whole genome sequencing is performed at a depth of about 1× or less.

6. The method of claim 1, further comprising labeling the organic material as genetically similar to the verified organic material when the genetic makeup of the organic material is verified.

7. The method of claim 2, wherein the profile of the reference sequence comprises one single polymorphism variant every 1 to 5K base pairs (bp) over the length of the genome of the verified organic material.

8. A method of verifying the genetic makeup of an organic material, comprising:
obtaining a biological sample of the organic material;
obtaining a pool of verified organic materials;
performing whole genome sequencing on each verified organic material from the pool;
identifying one or more single nucleotide polymorphism variants that are present within the verified organic materials and extracting any falsely identified single nucleotide polymorphisms to generate a reference sequence;
performing whole genome sequencing on the biological sample to generate one or more target sequence reads;
comparing the sequences of the one or more target sequence reads to the reference sequence; and
verifying the genetic makeup of the organic material when the one or more target sequence reads is substantially similar to that of the reference sequence.

9. The method of claim 8, wherein the pool of verified organic materials is comprised of heterozygous species.

10. The method of claim 9, wherein the pool comprises four or more samples of verified organic materials.

11. The method of claim 8, wherein the pool of verified organic materials is comprised of homozygous species.

12. The method of claim 11, wherein the pool comprises ten or more samples of verified organic materials.

13. The method of claim 8, wherein the reference sequence comprises a profile including one single polymorphism variant every 1 to 5K base pairs (bp) over the length of the genome of the verified organic material.

14. The method of claim 8, further comprising labeling the organic material as genetically similar to the verified organic material when the genetic makeup of the organic material is verified.

15. The method of claim 8, wherein the one or more target sequence reads is substantially similar to that of the reference sequence when the one or more target sequence reads comprises at least 90 percent identity to the reference sequence.

16. The method of claim 8, wherein the one or more target sequence reads is substantially similar to that of the reference sequence when the one or more target sequence reads comprises at least 95 percent identity to the reference sequence.

17. The method of claim 8, wherein the organic material is a plant.

18. The method of claim 8, wherein the organic material is an agricultural product.
